(19) United States
(12) Patent Application Publication
(10) Pub. No.: US 2013/0150712 A1
(43) Pub. Date: Jun. 13, 2013

(54) ASSEMBLIES AND METHODS

(71) Applicant: Smiths Medical International Limited, Ashford Kent (GB)

(72) Inventor: Stephen James Field, Kent (GB)

(73) Assignee: SMITHS MEDICAL INTERNATIONAL LIMITED, Ashford Kent (GB)

(21) Appl. No.: 13/760,406

(22) Filed: Feb. 6, 2013

Related U.S. Application Data
(63) Continuation-in-part of application No. 12/734,187, filed on Apr. 16, 2010, now Pat. No. 8,398,596.

(30) Foreign Application Priority Data
Nov. 15, 2007 (GB) 0722406.6

(51) Int. Cl. A61B 8/08; A61M 25/09; A61M 5/32

(52) U.S. Cl. CPC A61B 8/0841; A61M 5/3287; A61M 25/09
USPC 600/424

(57) ABSTRACT

A needle assembly comprises a metal needle (1) with a hub (13) and a marker in the form of a bubble-filled plastics rod (2) inserted within the needle. The rod (2) makes the assembly more visible under ultrasound observation when confirming correct placement of the needle (1) and is subsequently removed when correct placement has been confirmed. A fluid (3) is placed between the outside of the rod (2) and the inside of the needle (1) to improve acoustic coupling between the needle and the rod. Alternatively, a rod (72) is stretched during insertion and allowed to expand into close contact with the inside of the needle after insertion.

### CROSS-REFERENCES TO RELATED APPLICATIONS

This application is a Continuation-In-Part of application Ser. No. 12/734,187, filed on Apr. 16, 2010, which is based on PCT Application No. PCT/GB2008/003599, filed on Oct. 24, 2008, which in turn claims priority from GB Application No. 0722406.6, filed on Nov. 15, 2007.

### FIELD OF INVENTION

This invention relates to assemblies and methods, and is more particularly concerned with assemblies of needles or catheters having an insert extending along them.

### BACKGROUND OF INVENTION

Ultrasound scanners are used increasingly to help direct or check placement of catheters and other devices inserted in the body. Some of these devices are not normally very visible under ultrasound because of their shape, size or the fact that the material from which they are made has similar reflectance acoustic impedance to the tissue or body fluid within which they are inserted. Attempts have been made to increase the visibility of medico-surgical devices under ultrasound observation in various ways. Where the device, such as a needle, is of a metal the usual way of increasing its visibility is by modifying its surface, such as by forming grooves or indentations. A reflective coating may be applied to the device, such as incorporating bubbles, as described in WO98/19713 and EP0624342. Where the device is of a plastics material, such as a catheter of the kind described in GB2379610 the wall may include gas bubbles or a bubble-containing material may be incorporated in a stripe occupying only a part of the circumference. GB2400804 describes a similar catheter with several layers. U.S. Pat. No. 7,258,669 describes a catheter with a helical, gas-filled lumen extending along its length. WO9822022 describes an instrument with an inner stylet that may have an air void or a liquid containing microbubbles. DE102006051978 describes a bubble-filled rod inserted along the bore of a flexible plastics catheter to enhance visibility under ultrasound observation. WO2009/063166 describes a metal needle in which the ultrasound visibility is enhanced by a bubble-filled stylet extending within it or by a bubble-filled sleeve extending along its outside. A problem with a device having a bubble-filled insert extending along it is that a major part of the ultrasound energy may be reflected by the device itself, rather than by the insert, leading to a directional reflection, that is, a high signal at certain locations and orientations and a low signal at other locations and orientations

It is an object of the present invention to provide an alternative medico-surgical assembly and a method of its use.

### SUMMARY OF INVENTION

According to one aspect of the present invention there is provided a medico-surgical assembly including an outer tubular component, an inner component of a plastics material containing a plurality of gas bubbles within its thickness and a flowable substance between the outside of the inner component and the inside of the outer component to provide acoustic coupling between the outer and inner components so that during acoustic scanning acoustic energy passes through the outer component to the inner component, the bubbles in the inner component being selected to increase the amount of acoustic energy reflected by the inner component and hence by the assembly.

The flowable substance could be a body fluid or it could be liquid or gel applied to the outside of the inner component.

According to another aspect of the present invention there is provided a method of enhancing visibility of a catheter or needle under ultrasound observation comprising the steps of inserting the catheter or needle into a medium containing a liquid and subsequently sliding a bubble-filled stylet backwards and forwards within the catheter or needle so as to draw liquid from the medium into the bore of the catheter or needle and to distribute the liquid between the outside of the stylet and the inside of the needle or catheter such that the liquid forms a layer between the stylet and the needle that provides enhanced acoustic coupling between the stylet and the needle or catheter.

According to a third aspect of the present invention there is provided a method of enhancing visibility of a catheter or needle under ultrasound observation comprising the steps of inserting the catheter or needle into the body and subsequently sliding a bubble-filled stylet backwards and forwards within the catheter or needle so as to draw body fluid from the body into the bore of the catheter or needle and to distribute the fluid between the outside of the stylet and the inside of the needle or catheter such that the body fluid forms a layer of liquid between the stylet and the needle that provides enhanced acoustic coupling between the stylet and the needle or catheter.

According to a fourth aspect of the present invention there is provided a method of enhancing the visibility of a catheter or needle under ultrasound observation comprising the steps of applying a coating to the outside of a stylet of a bubble-filled plastics material, the coating being of a flowable material, inserting the stylet into the bore of a catheter or needle so that the flowable material forms a layer between the stylet and the catheter or needle that provides enhanced acoustic coupling between the stylet and the catheter or needle.

The gas bubbles preferably extend along substantially the entire length of the stylet but could be confined to one or more localised regions.

According to a fifth aspect of the present invention there is provided an assembly including an outer tubular component, an inner component of a stretchable material containing a plurality of gas bubbles within its thickness, the inner component having a natural external diameter greater than the internal diameter of the outer component so that the inner component is in compression within the outer component to provide acoustic coupling between the outer and inner components and so that during acoustic scanning acoustic energy passes through the outer component to the inner component, the bubbles in the inner component being selected to increase the amount of acoustic energy reflected by the inner component and hence by the assembly.

The inner component preferably has a bore extending along its length that is closed towards its forward end. The assembly may include an extension rod insertable within the bore of the inner component to stretch the inner component and reduce its external diameter during insertion within the outer component.

According to a sixth aspect of the present invention there is provided a method of enhancing visibility of a catheter or needle under ultrasound observation comprising the steps of providing a stylet having a bore extending along its length that is closed at its forward end, the stylet being of a stretchable material containing gas bubbles, inserting an extension rod into the bore of the stylet to stretch it longitudinally and reduce its external diameter from a first diameter greater than the internal diameter of the catheter or needle to a second diameter less than the internal diameter of the catheter or needle, inserting the stretched stylet into the bore of the catheter or needle to a desired position, withdrawing the extension rod and allowing the stylet to expand radially into close contact and acoustic coupling with the inside of the catheter or needle.

### BRIEF DESCRIPTION OF THE DRAWINGS

A needle assembly and its method of use, according to the present invention, will now be described, by way of example, with reference to the accompanying drawings, in which:

FIG. 1 is a cross-sectional side elevation view of the assembly to an enlarged scale;

FIG. 2 is an enlarged transverse sectional view along the line II-II of FIG. 1;

FIG. 3 illustrates the assembly in use;

FIG. 4 illustrates a method of applying an acoustic coupling substance to the assembly;

FIG. 5 shows an ultrasound scan of a needle alone in a phantom;

FIG. 6 shows an ultrasound scan of a needle and acoustically coupled bubble-filled stylet in the phantom; and

FIGS. 7A and 7B are cross-sectional side elevation views showing a needle with an alternative stylet at two stages of insertion.

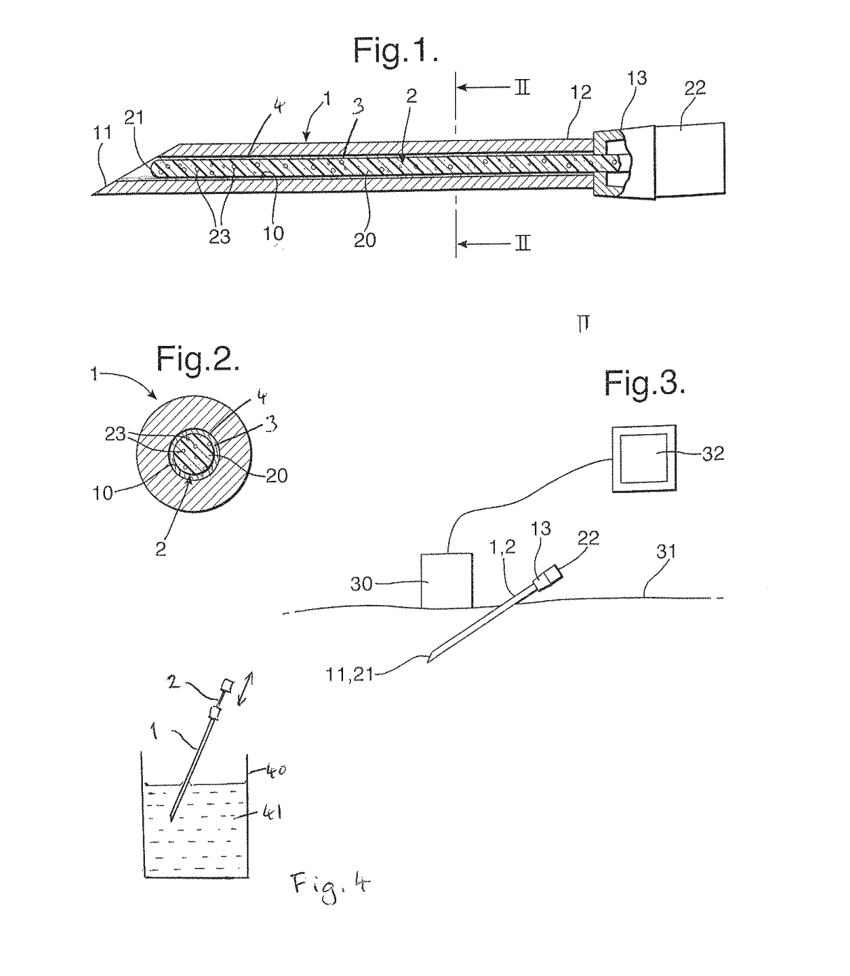

### DETAILED DESCRIPTION OF INVENTION

The assembly comprises an outer tubular component in the form of a conventional rigid, metal needle or cannula 1, an ultrasound marker in the form of an inner component or stylet 2 inserted within it and a layer of an acoustic coupling substance 3 between the stylet and the needle 1.

The needle 1 has a smooth external surface with a circular section and a coaxial bore 10 extending along its length. At its forward, patient end 11 the needle is bevelled to provide a sharp, penetrating tip. At its rear, machine end 12 the needle has a conventional hub 13 by which the needle can be attached to an external device such as the nose of a syringe or the like. The external diameter of the needle 1 is typically 0.5-2 mm and its length is typically 50-150 mm. The smooth surface of the needle 1 is such that it is not very visible by itself under ultrasound observation. Although the needle is shown as having an open, angled tip, it could be closed at its end and have a side opening close to its tip.

The stylet 2 comprises a solid, cylindrical rod 20 with a rounded forward or patient end 21 and is attached to a handle 22 at its rear end. The external diameter of the rod 20 is such that it is a free sliding fit within the bore 10 of the needle 1 with a small clearance or annular space 4 between the outside of the rod and the inside of the needle. When the handle 22 of the stylet 2 abuts the rear end of the hub 13, the patient end 21 lies substantially level with the patient end 11 of the needle. The rod 20 is extruded from a flexible plastics material, such as PEBA, nylon, PVC, polyethylene, polypropylene, polyester or polyurethane to which a foaming agent has been added so that the rod is filled with gas interstices in the form of bubbles 23 along its entire length. The size and density of the bubbles 23 are selected to ensure that the stylet 2 is highly echogenic. Typically the gas bubbles 23 have a size in the range 0.1μ to 300μ, preferably having a size in the range 1μ to 50μ and most preferably having a size in the range 5μ to 10. It will be appreciated that there are other ways of forming gas interstices such as by including gas-filled glass or polymer microparticles (such as Expancel) into the plastics material. The gas within the bubbles or interstices could be of any kind and could be a vacuum. Instead of having a multiplicity of gas bubbles, a single gas interstice could be provided by means of a hollow bore extending along the stylet and closed at both ends. The stylet 2 could instead be rigid, such as of ABS or styrenic materials. If the needle were not straight, the stylet would be preferably flexible. The stylet may have a round cross section but could be of other shapes. The outer tubular component need not be a metal needle but could be a catheter or cannula of a plastics material.

The assembly is completed by the layer of acoustic coupling substance 3 in the space 4 between the outside of the stylet 2 and the inside of the needle 1. The acoustic coupling substance 3 is a flowable substance such as a liquid, gel or paste and is chosen so that it will fill any annular space 4 between the outside of the stylet 2 and the inside of the needle 1. The nature of the substance 3 may vary according to the preferred manner in which it is applied. For example, it might be preferred to apply the substance to the outside of the stylet 2 before it is inserted into the bore 10 of the needle, or before it is entirely inserted into the needle (the tip of the stylet might be located in the rear end of the bore 10 before the acoustic coupling substance is applied). In such an arrangement, the acoustic coupling substance 3 would preferably be relatively thick or viscous so that it clings to the outside of the stylet 2. In this arrangement it might be preferred to use an acoustic gel similar to Aquasonic, Anagel or Sonogel. It will be appreciated that the acoustic coupling substance needs to be non-toxic, biologically compatible and compatible with any medication administered through the needle.

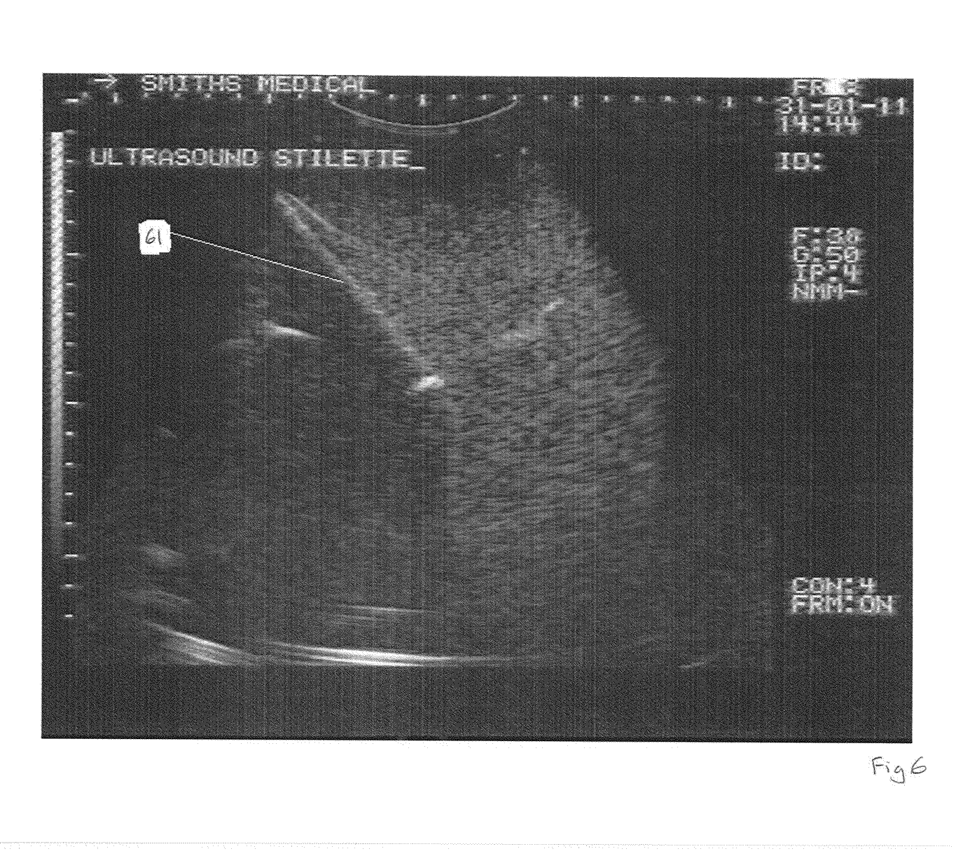

FIGS. 5 and 6 illustrate the effect of the acoustically-coupled bubble-filled stylet 2. FIG. 5 shows a scan picture taken of a conventional needle in a test phantom. This shows a faint image 51 from the needle extending down from the top left of the scan at an angle of about 120° to the vertical. The scan shows a much brighter, unwanted flare line 52 extending down from the tip 53 of the needle at the centre of the scan at an angle of about 210°. This flare is a result of the flat reflecting surface at the angled tip of the needle. By contrast, FIG. 6 shows the same needle 1 after insertion of the stylet 2. It can be seen that the image 61 of the needle 1 is more pronounced and also that the flare from the needle tip is dramatically reduced giving a much clearer image overall.

Instead of applying the acoustic coupling substance to the outside of the stylet 2, it could be flowed into the bore 10 of the needle 1 before the stylet 2 is fully inserted. One way of doing this would be to immerse the tip of the assembly into a bath 40 of an acoustic coupling liquid 41, as shown in FIG. 4, and then to push and pull the stylet 2 in and out of the needle 1. The liquid 41 will be drawn up into the bore 10 of the needle 1 to coat its inside as the stylet 2 is pulled out, and then the major part of the liquid is expelled as the stylet is pushed in. After this has been done a few times the liquid 41 will fill the annular space 4 between the outside of the stylet 2 and the needle 1 and held there by capillary action. This provides effective acoustic coupling between the needle 1 and the stylet 2.

This procedure could alternatively be carried out inside the body by inserting the needle 1 into the body and then by pushing and pulling the stylet 2 in and out of the needle a few times so that body fluid, such as blood, fills and coats the bore 10 of the needle and forms a layer between the stylet and the inside of the needle when the stylet is fully inserted.

The layer of acoustic coupling substance 3 between the stylet 2 and the needle 1 ensures that ultrasound energy incident on the needle passes efficiently through the wall of the needle and into the body of the stylet instead of being reflected at the material/air interface that would normally exist at the inner surface of the needle. In this way, a large part of the acoustic energy passes into the stylet 2 where it is reflected from the many gas bubbles 23 within it to give a high reflection signal over a wide range of viewing angles.

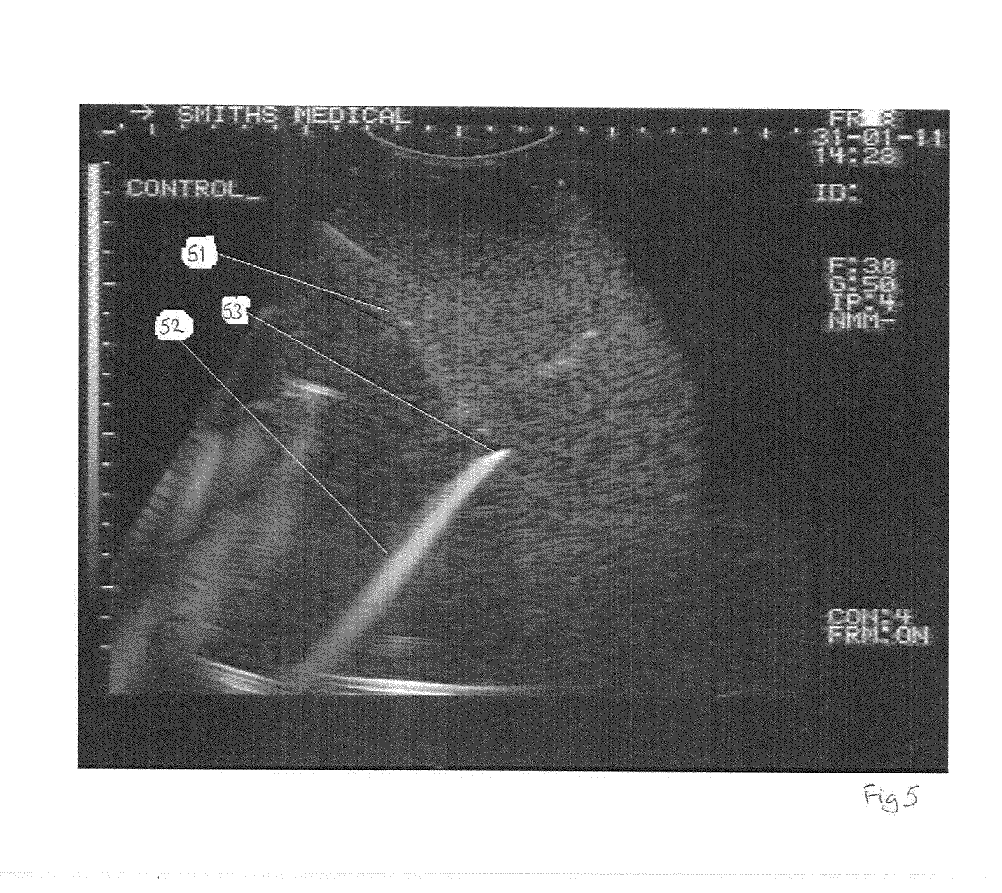

The stylet 2 can be used in several different ways. For example, the stylet 2 could be inserted in the needle 1 prior to insertion of the needle into the body so that the entire assembly of the needle and stylet is inserted at the same time. The scanning head 30 of an ultrasound instrument is then held against the skin 31 and the image of the underlying structure is viewed on a screen 32 of the instrument, as shown in FIG. 3. The stylet 2 makes the assembly highly reflective of ultrasound energy so the location of the assembly along its entire length is clearly visible on the screen 32. The clinician can then reposition the assembly as necessary until its tip 11, 21 or some other desired part of the needle (such as an eye) is in the desired position. Then he pulls out the stylet 2 to leave the needle 1 in position so that fluid can be flowed along the bore 10 of the needle, such as medication for administration to the patient or a sample taken from the patient via the needle. Instead, the needle could provide a path along which a catheter would be inserted, the needle subsequently being removed along the outside of the catheter, which is left in place in the body to enable prolonged or repeated administration of medication or sampling.

Alternatively, the needle could be inserted into the body without the echogenic stylet in position. When the needle is positioned at what is thought to be the correct position, the clinician would insert the echogenic stylet so that the correct positioning of the needle could then be confirmed by observation using the ultrasound scanner.

The stylet need not be a solid rod, as described, but could have an open or closed bore extending along its length or a wire or other member to alter its characteristics. Although it is generally preferable for the bubbles or other gas interstices to be provided along the entire length of the stylet, this is not essential and they could, instead, just be provided along a part of the length, such as towards the patient end.

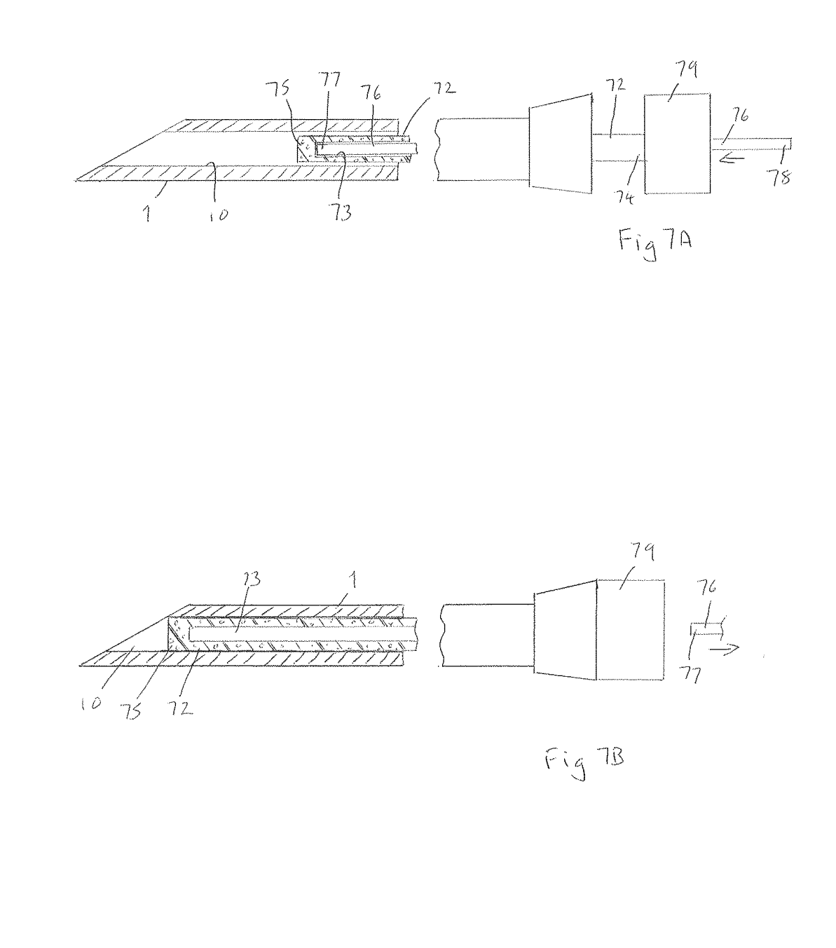

It is not essential that the acoustic coupling be achieved by means of a flowable substance between the stylet and the needle if a very close abutting fitting can be achieved between the outside of the stylet and the inside of the needle. One way of doing this is illustrated in FIGS. 7A and 7B. The inner component or stylet 72 is manufactured from a bubble-filled elastomeric or stretchable material with a natural external diameter that is slightly larger than the diameter of the bore through the outer component or needle 1. The stylet 72 has a bore 73 along its length that is open at its rear end 74 and closed at its forward, patient end 75. A stiff extension rod 76, such as of a metal or carbon fibre, extends slidably along the bore 73 of the stylet 72 with its forward end 77 abutting the closed forward end 75 of the bore 73 and its rear end 78 projecting from the handle 79 of the stylet 72. Before the stylet 72 is inserted in the bore 10 of the needle 1 the extension rod 76 is pushed into the stylet from its rear end 74 sufficiently to stretch the stylet longitudinally and thereby reduce its external radial diameter slightly from a first, natural diameter larger than the diameter or the bore to a second diameter less than that of the bore. This enables the stylet 72 to be inserted freely into the bore 10 of the needle 1, as shown in FIG. 7A. Once fully inserted, the extension rod 76 is pulled rearwardly out of the stylet 72, allowing it to relax and expand radially outwardly into firm contact with the inside of the needle 1, thereby establishing effective acoustic coupling between the stylet and the needle. The stylet 72 can be removed from the needle 1 simply by pulling its handle 79 since this will stretch it and reduce its external diameter sufficiently for it to slide out. Alternatively, the extension rod 76 could be reinserted to stretch the stylet 72 and reduce its external diameter.

It will be appreciated that the present invention is not confined to visual ultrasound observation since it could have advantages when used with an ultrasound scanner that provides, for example an audible feedback to the user, such as an increasing tone when positioned in alignment with an ultrasound reflective device.

1. A needle assembly including a hollow metal needle, wherein the assembly includes a plastics rod removably mounted within the needle along its length and containing a plurality of gas bubbles within the thickness of the rod along its length such that the rod is visible under ultrasound observation and such that the ultrasound visibility of the assembly with the rod is greater that that of the needle alone.

2. An assembly according to claim 1, wherein the gas bubbles extend along substantially the entire length of the rod.

3. An assembly according to claim 1, wherein the gas bubbles have a size between 0.1μ and 300μ.

4. An assembly according to claim 3, wherein the gas bubbles have a size between approximately 5μ and 10μ.

5. An assembly according to claim 1, wherein the assembly includes a flowable substance between the outside of the rod and the inside of the needle to provide acoustic coupling between the rod and needle so that during acoustic scanning acoustic energy passes through the needle to the rod.

6. An assembly according to claim 5, wherein the flowable substance is a body fluid.

7. An assembly according to claim 5, wherein the flowable substance is a liquid or gel applied to the outside of the rod.

8. A method of enhancing visibility of a catheter or needle under ultrasound observation comprising the steps of: inserting the catheter or needle into a medium containing a liquid and subsequently sliding a bubble-filled stylet backwards and forwards within the catheter or needle so as to draw liquid from the medium into the bore of the catheter or needle and to distribute the liquid between the outside of the stylet and the inside of the needle or catheter such that the liquid forms a layer between the stylet and the needle that provides enhanced acoustic coupling between the stylet and the needle or catheter.

9. A method of enhancing visibility of a catheter or needle under ultrasound observation comprising the steps of: inserting the catheter or needle into the body and subsequently sliding a bubble-filled stylet backwards and forwards within the catheter or needle so as to draw body fluid from the body into the bore of the catheter or needle and to distribute the fluid between the outside of the stylet and the inside of the needle or catheter such that the body fluid forms a layer of liquid between the stylet and the needle that provides enhanced acoustic coupling between the stylet and the needle or catheter.

10. A method of enhancing the visibility of a catheter or needle under ultrasound observation comprising the steps of: applying a coating to the outside of a stylet of a bubble-filled plastics material, the coating being of a flowable material, inserting the stylet into the bore of a catheter or needle so that the flowable material forms a layer between the stylet and the catheter or needle that provides enhanced acoustic coupling between the stylet and the catheter or needle.

11. An assembly including an outer tubular component, an inner component of a stretchable material containing a plurality of gas bubbles within its thickness, the inner component having a natural external diameter greater than the internal diameter of the outer component so that the inner component is in compression within the outer component to provide acoustic coupling between the outer and inner components and so that during acoustic scanning acoustic energy passes through the outer component to the inner component, the bubbles in the inner component being selected to increase the amount of acoustic energy reflected by the inner component and hence by the assembly.

12. An assembly according to claim 11, wherein the inner component has a bore extending along its length that is closed towards its forward end, and wherein the assembly includes an extension rod insertable within the bore of the inner component to stretch the inner component and reduce its external diameter during insertion within the outer component.

13. A method of enhancing visibility of a catheter or needle under ultrasound observation comprising the steps of: providing a stylet having a bore extending along its length that is closed at its forward end, the stylet being of a stretchable material containing gas bubbles, inserting an extension rod into the bore of the stylet to stretch it longitudinally and reduce its external diameter from a first diameter greater than the internal diameter of the catheter or needle to a second diameter less than the internal diameter of the catheter or needle, inserting the stretched stylet into the bore of the catheter or needle to a desired position, withdrawing the extension rod and allowing the stylet to expand radially into close contact and acoustic coupling with the inside of the catheter or needle.
